(19) United States
(12) Patent Application Publication
(10) Pub. No.: US 2026/0069885 A1
(43) Pub. Date: Mar. 12, 2026

(54) SKIN TREATMENT DEVICE

(71) Applicant: Shenzhen Ulike Smart Electronics Co., Ltd., Shenzhen (CN)

(72) Inventor: Lijun TAN, Shenzhen (CN)

(21) Appl. No.: 19/389,316

(22) Filed: Nov. 14, 2025

Related U.S. Application Data
(63) Continuation-in-part of application No. PCT/CN2025/082365, filed on Mar. 13, 2025.

(30) Foreign Application Priority Data
Mar. 15, 2024 (CN) 202420541129.2
Apr. 11, 2024 (CN) 202420749054.7

(51) Int. Cl. A61N 5/06; A61N 5/00

(52) U.S. Cl. CPC A61N 5/0616; A61N 2005/007; A61N 2005/0632; A61N 2005/0654; A61N 2005/0666; A61N 2005/0667

(57) ABSTRACT

A skin treatment device is disclosed in the present application. The skin treatment device includes a housing and a light emitting assembly, the housing includes a light emitting portion. The light emitting assembly is disposed in the housing; a light emitting channel is formed between the light emitting assembly and the light emitting portion, the light emitting channel is provided with a partition member, the partition member divides the light emitting channel into a first sub-light emitting channel and a second sub-light emitting channel, the light emitting assembly comprises a first light source and a second light source, the first light source is configured to generate light that emits toward the light emitting portion through the first sub-light emitting channel, the second light source is configured to generate light that emits toward the light emitting portion through the second sub-light emitting channel.

The present application claims the benefit and priority to Chinese Patent Application Serial No. 202420541129.2, filed on Mar. 15, 2024, entitled “light source assembly applied to skin treatment device and skin treatment device” and Chinese Patent Application Serial No. 202420749054.7, filed on Apr. 11, 2024, entitled “skin treatment device”, and the content of which is hereby fully incorporated by reference into the present application.

### FIELD

The present application is generally related to the field of skin treatment technology and, more specifically to a skin treatment device.

### BACKGROUND

Skin treatment devices such as skin rejuvenation devices or depilation instruments usually apply multiple light sources that work in conjunction with each other to produce different types of light to treat the skin. Due to the layout requirement within the skin treatment device, the requirements may be to arrange one or more structures between the light source and the light outlet portion of the skin treatment device, the one or more structures may be cold packs, refrigerators, heat pipes, so that there is a distance between the light source and the light outlet portion, and then the light generated by the light source needs to pass through the distance to be emitted from the light outlet portion, resulting in the light being prone to damage in the propagation process. Any one of a plurality of different types of light sources is eccentric with respect to the center of the light exit channel, so that the light energy can be transmitted to the light outlet surface more difficultly.

### SUMMARY OF THE INVENTION

The present application provides a skin treatment device to solve the above technical problems.

The present application accomplishes the above purposes by the following technical solutions.

The present application provides a skin treatment device, the skin treatment device includes a housing and a light emitting assembly, the housing includes a light emitting portion. The light emitting assembly is disposed in the housing; wherein a light emitting channel is formed between the light emitting assembly and the light emitting portion, the light emitting channel is provided with a partition member, the partition member divides the light emitting channel into a first sub-light emitting channel and a second sub-light emitting channel, both the first sub-light emitting channel and the second sub-light emitting channel are opposite to the light emitting portion, the light emitting assembly comprises a first light source and a second light source, the first light source is opposite to the first sub-light emitting channel and configured to generate light that emits toward the light emitting portion through the first sub-light emitting channel, the second light source is opposite to the second sub-light emitting channel and configured to generate light that emits toward the light emitting portion through the second sub-light emitting channel; a type of the first light source is different from a type of the second light source.

In the skin treatment device provided in the present application, a light emitting assembly of the skin treatment device is disposed in the housing. The light emitting channel is formed between the light emitting assembly and the light emitting portion, the light emitting channel is provided with a partition member, the partition member divides the light emitting channel into a first sub-light emitting channel and a second sub-light emitting channel. Both the first sub-light emitting channel and the second sub-light emitting channel are opposite to the light emitting portion. The first light source is opposite to the first sub-light emitting channel and configured to generate light that emits toward the light emitting portion through the first sub-light emitting channel, while the second light source is opposite to the second sub-light emitting channel and configured to generate light that emits toward the light emitting portion through the second sub-light emitting channel. As such, the partition member can reduce the light generated by the first light source from entering the second sub-light emitting channel during its propagation in the first sub-light emitting channel, and also reduce the light generated by the second light source from entering the first sub-light emitting channel during its propagation in the second sub-light emitting channel, thereby helping to improve light concentration and reduce light loss caused by offset and divergence in the light emitting channel.

On one hand, an embodiment of the present application provides a light source assembly applied to a skin treatment device, the light source assembly includes multiple light sources and a reflective member, the reflective member includes a light outlet, the light outlet is configured for emitting light from the multiple light sources; wherein any two adjacent light sources of the multiple light sources are spaced apart within the reflective member; and wherein the reflective member is further provided with a protrusion portion extending toward a gap between the any two adjacent light sources.

In summary, because the relevant technology places multiple lamps in a reflective cup, the light emitted by the multiple lamps will interfere with each other and the multiple lamps will block part of the reflected light from each other, which results in the energy of the light emitted by the lamps being weakened. The present application, by providing a protrusion portion extending from the gap between any two adjacent light sources of the multiple light sources in a reflective member, makes the light generated by the multiple light sources in the reflective member not block each other, and the light generated by the multiple light sources not interfering with each other, so that the light energy generated by multiple light sources does not weaken, to further enhance the output energy of the light source assembly, improve the beauty effect, and meet the needs of users.

### BRIEF DESCRIPTION OF THE DRAWINGS

In order to more clearly illustrate the technical solutions of the present application, the following will briefly introduce the accompanying drawings in the embodiments, it should be understood that the following drawings only illustrate certain embodiments of the present application and should not be construed as limiting the scope, for those of ordinary skill in the art, other relevant drawings may be obtained from these drawings without creative labor.

FIG. 1 is a schematic diagram of an embodiment of a skin treatment device according to the present application.

FIG. 2 is a schematic diagram of another view of the skin treatment device shown in FIG. 1.

FIG. 3 is a sectional view of the skin treatment device shown in FIG. 2 along line III-III.

FIG. 4 is a partial schematic diagram of the skin treatment device shown in FIG. 1.

FIG. 5 is a schematic diagram of another view of the skin treatment device shown in FIG. 1.

FIG. 6 is a sectional view of the skin treatment device shown in FIG. 5 along line VI-VI.

FIG. 7 is a perspective view of an embodiment of a light source assembly of the present application.

FIG. 8 is a schematic diagram of a reflective member shown in FIG. 7.

FIG. 9 is a front view shown in FIG. 7.

FIG. 10 is a schematic diagram of another light source assembly of the present application.

FIG. 11 is a front view shown in FIG. 10.

FIG. 12 is a front view of another embodiment of a light source assembly of the present application.

FIG. 13 is a perspective view of another embodiment of a skin treatment device of the present application.

FIG. 14 is an exploded view shown in FIG. 13.

FIG. 15 is a partial cross-sectional view shown in FIG. 13.

FIG. 16 is a partial schematic view of a mounting bracket shown in FIG. 15.

### DETAILED DESCRIPTION OF THE INVENTION

In order for those skilled in the art to better understand the technical solution of the present application, the technical solutions in the embodiments of the present application will be clearly and completely described in the following with reference to the accompanying drawings in the embodiments of the present application. The described embodiments are only a part of the embodiments of the present application, and not all of them. All other embodiments, which can be made by those skilled in the art based on the embodiments of the application without making any inventive effort, are intended to be within the scope of the application.

The technical solutions in the embodiments of the present application will be clearly described below in conjunction with the accompanying drawings in the embodiments of the present application.

The skin treatment device of related technology usually compensates for optical losses by increasing the output power, but this increases power consumption and poses a risk of overheating.

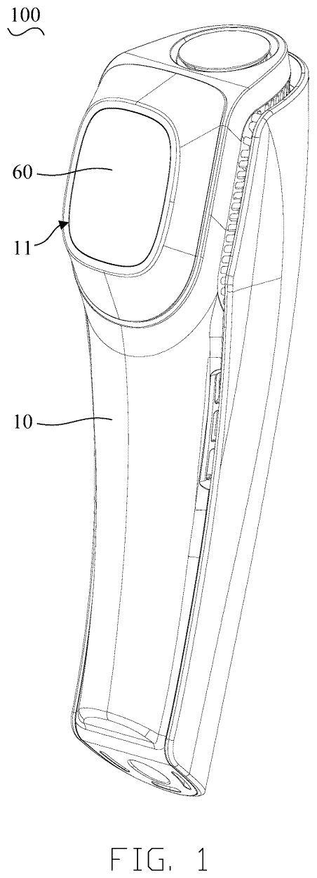

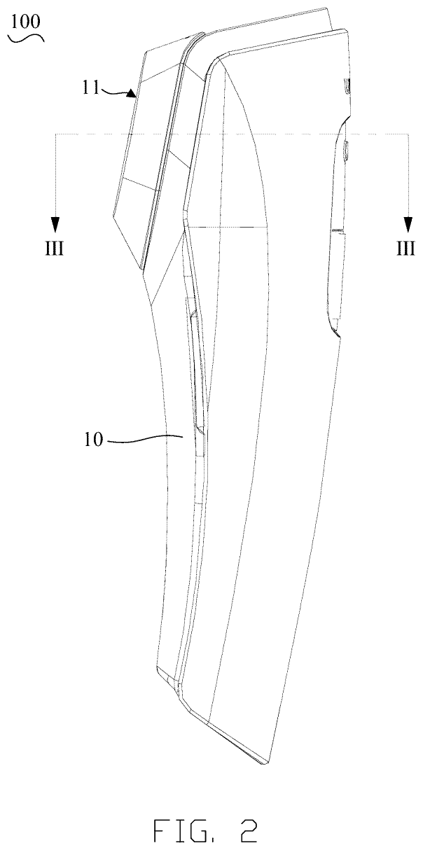

FIGS. 1 to 3 illustrate a skin treatment device 100 in accordance with an embodiment of the present application. The skin treatment device 100 is a device for adjusting and improving the body and skin conditions in accordance with the physiological functions of the human body, and it has effects of whitening, tendering, removing spots, removing wrinkles, removing hair. Based on optics, different effects can be achieved by irradiating the skin with special wavelengths of light or different types of light. For example, the more widely used ones are Intense Pulsed Light (IPL), LED light and laser.

In some embodiments, the skin treatment device 100 may be a beauty instrument, for example, the skin treatment device 100 may be a skin rejuvenation device, a depilation instrument, or other types.

In some embodiments, the skin treatment device 100 includes a housing 10 and a light emitting assembly 20, and the light emitting assembly 20 is disposed within the housing 10. The housing 10 has a light emitting portion 11, which facilitates the light generated by the light emitting assembly 20 to emit from the housing 10 toward the skin of the user.

In some embodiments, the light emitting portion 11 may be a light emitting opening, a light emitting hole, or other structures disposed on the housing 10.

In some embodiments, the light emitting portion 11 may also be formed by some light-transmissive areas on the housing 10. For example, at least a part of the housing 10, such as the head shell, may be made of light-transmissive material, with a light-shielding layer partially disposed on the head shell to form the light emitting portion 11 where the head shell is not provided with a light-shielding layer.

In some embodiments, the light emitting assembly 20 includes a first light source 21 and a second light source 22, the first light source 21 and the second light source 22 are of different types, this helps the light emitting assembly 20 generate different types of light through the first light source 21 and the second light source 22, to have different penetration power and mechanisms of action on different layers of skin, which can act more deeply on various skin layers, thereby improving the overall treatment effect.

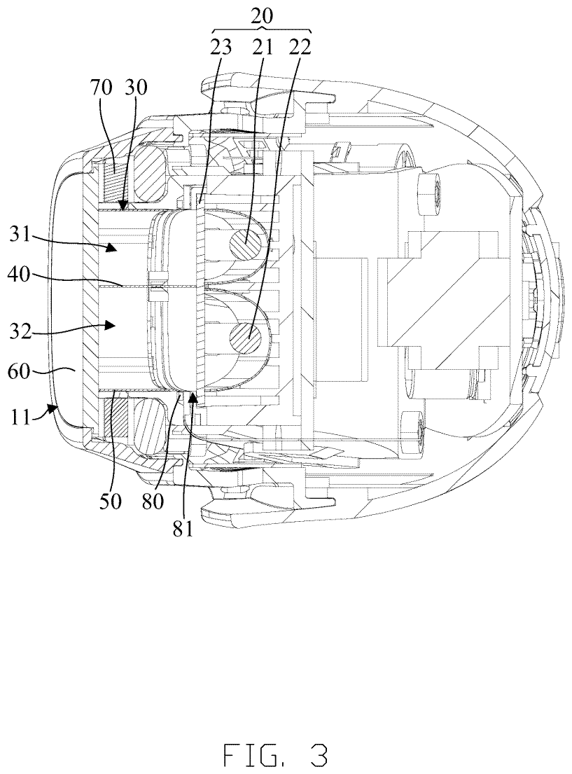

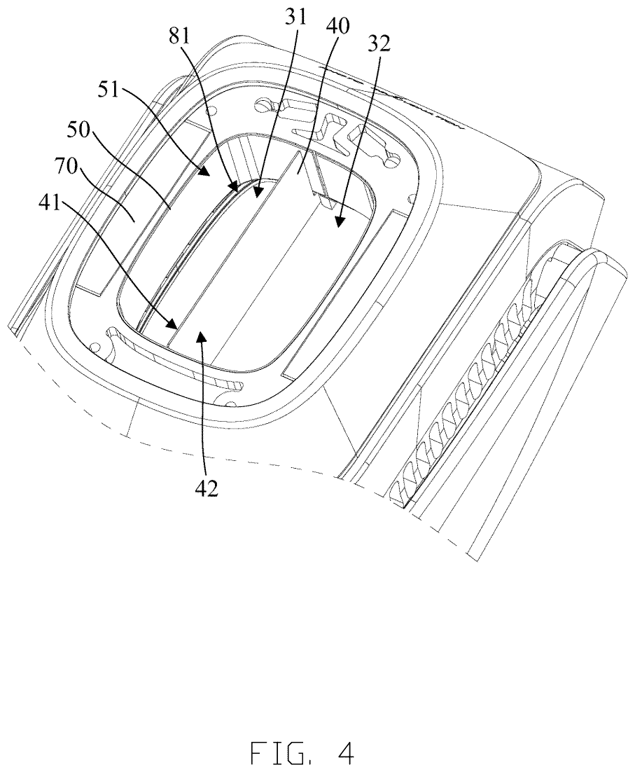

Referring to FIGS. 3 and 4, in some embodiments, a light emitting channel 30 is formed between the light emitting assembly 20 and the light emitting portion 11, the light emitting channel 30 is provided with a partition member 40, the partition member 40 divides the light emitting channel 30 into a first sub-light emitting channel 31 and a second sub-light emitting channel 32. Both the first sub-light emitting channel 31 and the second sub-light emitting channel 32 are opposite to the light emitting portion 11. The first light source 21 is opposite to the first sub-light emitting channel 31 and configured to generate light that emits toward the light emitting portion 11 through the first sub-light emitting channel 31, while the second light source 22 is opposite to the second sub-light emitting channel 32 and configured to generate light that emits toward the light emitting portion 11 through the second sub-light emitting channel 32.

As such, the partition member 40 can help to reduce the light generated by the first light source 21 from entering the second sub-light emitting channel 32 during its propagation in the first sub-light emitting channel 31, and also reduce the light generated by the second light source 22 from entering the first sub-light emitting channel 31 during its propagation in the second sub-light emitting channel 32, thereby helping to improve light concentration and reduce light loss caused by offset and divergence in the light emitting channel 30. In addition, the skin treatment device 100 may not need to increase light output power to compensate for light loss, which may reduce energy consumption of the skin treatment device 100.

In some embodiments, the partition member 40 may be substantially flat-shaped, for example, the partition member 40 may be a partition plate, this helps reduce the space occupied by the partition member 40 within the light emitting channel 30.

In some embodiments, the first light source 21 may be a lamp, and the second light source 22 may be a lamp.

In some embodiments, the first light source 21 may be a halogen light source, and the second light source 22 may be a pulsed light source, these two types of light sources can provide targeted treatment effects for different skin problems. For example, the halogen light source may be used for basic care and heating, while the pulsed light source can be used for deep cleaning or activating collagen regeneration, these two types of light sources help achieve multi-functional and multi-effect skin treatment effects of the skin treatment device 100.

In some embodiments, the halogen light source may be used to generate Near-Infrared (NIR) light or other light.

In some embodiments, the pulsed light source may be used to generate Intense Pulsed Light (IPL), Direct Pulse Light (DPL), or other light.

In some embodiments, the light emitting assembly 20 may also include more than three different types of light sources. For example, the light emitting assembly 20 may also include a third light source, a fourth light source, a fifth light source, and so on. Correspondingly, the number of the sub-light emitting channels may match the number of the light sources. For example, the light emitting channel 30 may also include a third sub-light emitting channel, a fourth sub-light emitting channel, a fifth sub-light emitting channel, and so on. The third light source may be opposite to the third sub-light emitting channel and configured to generate light that emits toward the light emitting portion 11 through the third sub-light emitting channel. The fourth light source may be opposite to the fourth sub-light emitting channel and configured to generate light that emits toward the light emitting portion 11 through the fourth sub-light emitting channel. The fifth light source may be opposite to the fifth sub-light emitting channel and configured to generate light that emits toward the light emitting portion 11 through the fifth sub-light emitting channel.

In some embodiments, a light guiding ring 50 may be disposed within the light emitting channel 30, the partition member 40 is located at the inner ring space of the light guiding ring 50. The partition member 40 includes a first surface 41 and a second surface 42, the first surface 41 and the second surface 42 face away from each other. The first sub-light emitting channel 31 is located between the inner ring surface 51 of the light guiding ring 50 and the first surface 41, while the second sub-light emitting channel 32 is located between the inner ring surface 51 of the light guiding ring 50 and the second surface 42. Therefore, the light guiding ring 50 helps guide the light generated by the first light source 21 and the second light source 22 to propagate within the light emitting channel 30, and also helps make the light more concentrated during propagation, thereby reducing light loss.

In some embodiments, the light guiding ring 50 may be generally circular ring-shaped, elliptical ring-shaped, square ring-shaped, or other shapes.

In some embodiments, the light guiding ring 50 may be a continuous ring structure. The light guiding ring 50 may also be a ring structure where the front and rear ends are not connected, or in other words, the ring surface of the light guiding ring 50 has a notch.

In some embodiments, a reflective layer (not shown in the figures) may be disposed on the inner ring surface 51 of the light guiding ring 50, and the reflective layer may be located in the first sub-light emitting channel 31 and/or the second sub-light emitting channel 32.

For example, the reflective layer may be located in the first sub-light emitting channel 31, the reflective layer may enhance the reflectivity of the inner ring surface 51 of the light guiding ring 50 to light, helping to reflect light that becomes offset and divergent during propagation in the first sub-light emitting channel 31, improving light concentration and reducing light loss within the first sub-light emitting channel 31, helping to enhance the light efficiency of the light emitting assembly 20.

For another example, the reflective layer may be located in the second sub-light emitting channel 32, the reflective layer may enhance the reflectivity of the inner ring surface 51 of the light guiding ring 50 to light, helping to reflect light that becomes offset and divergent during propagation in the second sub-light emitting channel 32, improving light concentration and reducing light loss within the second sub-light emitting channel 32, helping to enhance the light efficiency of the light emitting assembly 20.

For another example, the reflective layer may be located in the first sub-light emitting channel 31 and the second sub-light emitting channel 32, which can reduce light loss within both channels.

In some embodiments, a reflective layer may be disposed on the first surface 41 and/or the second surface 42 of the partition member 40.

For example, a reflective layer may be disposed on the first surface 41 of the partition member 40, the reflective layer can enhance the reflectivity of the first surface 41 to light, helping to reflect light that becomes offset and divergent during propagation in the first sub-light emitting channel 31, improving light concentration and reducing light loss within the first sub-light emitting channel 31.

For another example, a reflective layer may be disposed on the second surface 42 of the partition member 40, the reflective layer can enhance the reflectivity of the second surface 42 to light, helping to reflect light that becomes offset and divergent during propagation in the second sub-light emitting channel 32, improving light concentration and reducing light loss within the second sub-light emitting channel 32.

For another example, the reflective layers are disposed on both the first surface 41 and the second surface 42 of the partition member 40, it helps reduce light loss within both the first sub-light emitting channel 31 and the second sub-light emitting channel 32.

In some embodiments, the reflective layer of the partition member 40 and the reflective layer of the light guiding ring 50 may be of the same or different types.

In some embodiments, the reflective layer may be sprayed on the partition member 40 and the light guiding ring 50, or the reflective layer may be plated on the partition member 40 and the light guiding ring 50.

In some embodiments, the light emitting assembly 20 may also include a light filter 23, the light filter 23 is disposed in the light paths of the first light source 21 and the second light source 22. The light filter 23 helps to filter harmful or ineffective light, ensuring that light of appropriate intensity and wavelength acts on the skin, and to avoid overly strong light waves from causing harm to the skin, such as thermal damage or burns.

In some embodiments, the partition member 40 may abut against the surface of the light filter 23 that faces away from the first light source 21 or the second light source 22. As such, in a first aspect, it helps to reduce the light generated by the first light source 21 from scattering into the second sub-light emitting channel 32 after passing through the light filter 23, thereby helping to increase the amount of light entering the first sub-light emitting channel 31; in a second aspect, it helps to reduce the light generated by the second light source 22 from scattering into the first sub-light emitting channel 31 after passing through the light filter 23, thereby helping to increase the amount of light entering the second sub-light emitting channel 32; in a third aspect, it helps to improve the compactness between the partition member 40 and the light filter 23, so that the relative positions of the partition member 40 and the light filter 23 are not prone to be shifted due to vibration or other reasons.

In some embodiments, the partition member 40 may extend from the light filter 23 to the light emitting portion 11, which helps to form a continuous isolation structure along the light propagation direction in the light emitting channel 30, so as to be able to better separate the first sub-light emitting channel 31 and the second sub-light emitting channel 32, which helps to maintain the concentration of the light within the first sub-light emitting channel 31 and the second sub-light emitting channel 32, thereby helping to reduce the loss of light.

In some embodiments, the light guiding ring 50 and the partition member 40 may be an integrally molded structure. For example, the light guiding ring 50 and the partition member 40 may be integrally molded using a mold, which can reduce the number of parts in the skin treatment device 100 and facilitate the assembly of the light guiding ring 50 and partition member 40 into the housing 10. In addition, the integral molded structure can enhance the connection strength and stability between the light guiding ring 50 and the partition member 40, reduce the risk of device failure due to loosening or separation of parts, and ensure the reliability and durability of the device during use.

In some embodiments, the light emitting portion 11 may be provided with a cold compress member 60 for cold compressing the skin. The cold compress member 60 may be located in the light path of the first light source 21 and the second light source 22, so that the light generated by the first light source 21 and the second light source 22 can be directed to the skin to be treated through the cold compress member 60, and the cold compress member 60 can reduce or even eliminate the pain or burning sensation generated when the light acts on the skin.

In some embodiments, the cold compress member 60 may be sapphire, the cold compress member 60 may be generally block-shaped, plate-shaped, or other shapes.

In some embodiments, the side of the partition member 40 facing away from the light filter 23 may abut against the cold compress member 60, which may reduce light generated by the first light source 21 from scattering from the first sub-light emitting channel 31 to the second sub-light emitting channel 32, also reduce light generated by the second light source 22 from scattering from the second sub-light emitting channel 32 to the first sub-light emitting channel 31, and also improve the compactness between the partition member 40 and the light filter 23, so that the partition member 40 and the light filter 23 are not prone to be shifted in relative position due to vibration or other reasons.

In some embodiments, there may be a gap between the side of the partition member 40 backing away from the light filter 23 and the cold compress member 60, this gap helps to adapt to the manufacturing tolerances of the structure and facilitate assembly without affecting or significantly affecting light loss.

In some embodiments, the surfaces of the light guiding ring 50 and the light filter 23 backing away from the first light source 21 or the second light source 22 may either abut against or have a gap 81, this gap helps to adapt to the manufacturing tolerances of the structure and facilitate assembly without affecting or significantly affecting light loss. Similarly, this helps reduce light generated by the first light source 21 from scattering into the second sub-light emitting channel 32 after passing through the light filter 23 and helps reduce light generated by the second light source 22 from scattering into the first sub-light emitting channel 31 after passing through the light filter 23. In addition, when the light guiding ring 50 abuts against the light filter 23, it helps improve the compactness of the light guiding ring 50 and the light filter 23.

In some embodiments, the side of the light guiding ring 50 backing away from the light filter 23 may abut against the cold compress member 60, which can help to reduce light generated by the first light source 21 from scattering from the first sub-light emitting channel 31 to the second sub-light emitting channel 32, also help reduce light generated by the second light source 22 from scattering from the second sub-light emitting channel 32 to the first sub-light emitting channel 31, and also help improve the compactness between the light guiding ring 50 and the cold compress member 60, so that the light guiding ring 50 and the cold compress member 60 are not prone to be shifted in relative position due to vibration or other reasons.

In some embodiments, there may be a gap between the side of the light guiding ring 50 facing away from the light filter 23 and the cold compress member 60, this gap helps to adapt to the manufacturing tolerances of the structure and facilitate assembly without affecting or significantly affecting light loss.

In some embodiments, the skin treatment device 100 may also include a supplementary light source 80 disposed within the housing 10. The light from the supplementary light source 80 can emit into the light emitting channel 30 through the gap 81 between the light guiding ring 50 and the light filter 23, for example, the light from the supplementary light source 80 may be emitted through the gap 81 to the first sub-light emitting channel 31 and the second sub-light emitting channel 32, which helps to improve the light quantity and emission uniformity of the skin treatment device 100.

In some embodiments, the supplementary light source 80 may surround the light emitting channel 30, which helps to improve the light quantity and emission uniformity of the skin treatment device 100 while avoiding blocking the light paths of the first light source 21 and the second light source 22, thereby reducing the light loss of the first light source 21 and the second light source 22.

In some embodiments, the supplementary light source 80 may be a light-emitting diode (LED) light source. For example, the supplementary light source 80 may include a substrate and multiple LED chips, the substrate may be a flexible circuit board or other type, and the multiple LED chips may be spaced apart on the substrate.

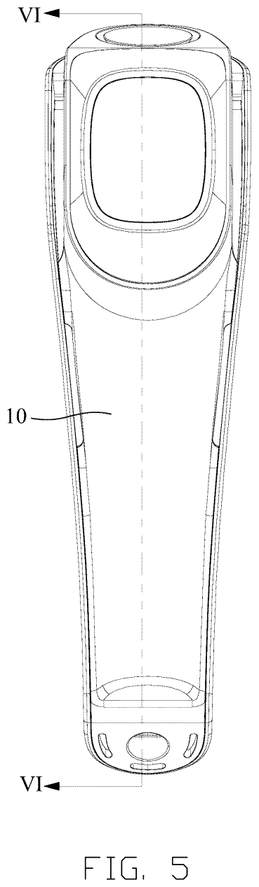

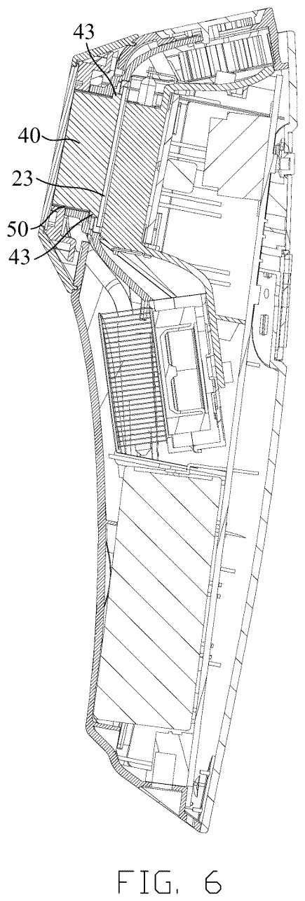

Referring to FIGS. 5 and 6, in some embodiments, the partition member 40 may include a support portion 43 extending into the gap 81. One end of the support portion 43 abuts against the light filter 23, and another end of the support portion 43 abuts against the light guiding ring 50, thereby maintaining the gap between the light guiding ring 50 and the light filter 23. Therefore, the support portion 43 can space the light guiding ring 50 and the light filter 23, so that the light guiding ring 50 and the light filter 23 are not easily pressed against each other due to vibration or other reasons, and ensures that the relative positions between the light guiding ring 50 and the light filter 23 are fixed, thereby helping to form and maintain a gap 81 between the light guiding ring 50 and the light filter 23, and helping the light produced by the supplementary light source 80 to enter the light emitting channel 30.

Referring to FIGS. 3 and 4, in some embodiments, the skin treatment device 100 may also include a refrigerator 70, the cooling surface of the refrigerator 70 may be provided on the surface of the cold compress member 60 toward the light emitting assembly 20, so that the cold generated by the refrigerator 70 can be transferred to the cold compress member 60 for cold compressing the skin.

In some embodiments, the cooling surface of the refrigerator 70 may be located at the outer periphery of the first sub-light emitting channel 31 or the second sub-light emitting channel 32, which can help to avoid the refrigerator 70 from blocking light within the first sub-light emitting channel 31 or the second sub-light emitting channel 32.

In some embodiments, the heating surface of the refrigerator 70 may be thermally connected to the heat sink of the skin treatment device 100 through a heat pipe, so that the heat sink can dissipate heat from the heating surface of the refrigerator 70.

In some embodiments, the refrigerator 70 may be a thermocouple refrigerator, a semiconductor refrigerator, a laser refrigerator, or other structure.

It can be understood that with the development of technology, more and more beauty products such as skin rejuvenation devices or depilation instruments, etc., are being used by people. Skin rejuvenation devices or depilation instruments in related technology mostly use a single light source for hair removal, whitening, and other beauty treatments. These beauty products generally include a reflective cup and a lamp, and the single lamp is disposed in the reflective cup. The reflective cup has a reflective surface and forms a light outlet, so that through the reflection and focusing effect of the reflective surface, the light energy output from the lamp through the light outlet can meet the requirements for beauty treatment. The higher the light energy emitted from the light outlet in beauty products, the more significant the hair removal or beauty effect. Therefore, to achieve more significant hair removal or beauty effects, beauty products in related technology would set multiple lamps in the reflective cup to enhance light energy output and improve beauty effects.

However, through creative labor, the inventors discovered that in some scenarios, the reflection effect of the reflective surface on the reflective cup causes interference between light from multiple lamps, and multiple lamps block some reflected light from each other, leading to weakened light energy output from the lamps, resulting in final light energy output that cannot meet user requirements. In order to overcome these problems, another embodiment of the present application provides a light source assembly applied to skin treatment device and a skin treatment device.

In the embodiments of the present application, the light source assembly 400 is an assembly applied to the skin treatment device 1, the skin treatment device 1 may include devices such as skin rejuvenation devices or depilation instruments that can treat the skin. The light source assembly 400 includes multiple light sources 110, for example, two light sources, three light sources, or more light sources 110. In the embodiment, two light sources are used as an example to illustrate multiple light sources 110. When there is no conflict, the features of the skin treatment device 1 provided in the embodiment present can be combined with the features of the above-mentioned skin treatment device 100.

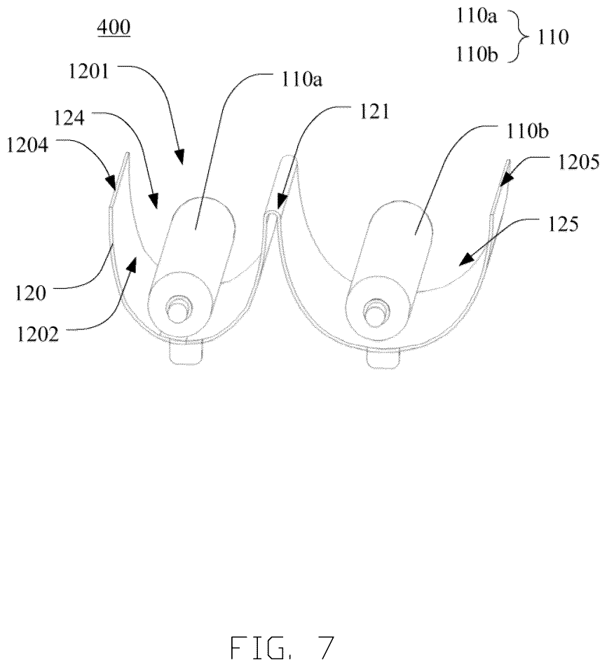

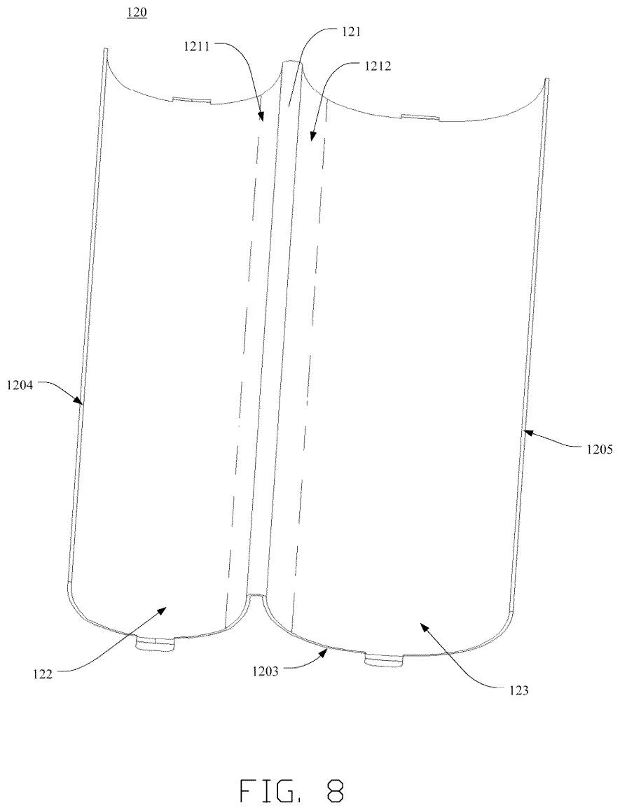

Referring to FIGS. 7 and 8, the embodiment of the present application provides a light source assembly 400 applied to skin treatment device, the light source assembly 400 includes multiple light sources 110 and a reflective member 120.

Specifically, in the embodiment, two light sources are used as an example to illustrate multiple light sources 110, in other words, in the embodiment, two light sources includes a first light source 110a and a second light source 110b, this description is not intended to limit the embodiment of the present application. The first light source 110a and the second light source 110b may be IPL lamps that can emit intense pulsed light or halogen lamps.

In some embodiments, the reflective member 120 may be a single reflective member. In other embodiments, the reflective member 120 may also refer to two separately set reflective members, meaning that the first light source and the second light source each correspond to a reflective member, two reflective members approach each other at adjacent end to form a protrusion, the adjacent ends of the two reflective members can be directly connected, connected through a connecting member, or stacked along the light emitting direction to reduce the risk of light leakage. For simplicity of description, the following explanation uses a single reflective member 120 as an example.

The reflective member 120 defines a light outlet 1201, and the light outlet 1201 is used for emitting light from the light sources 110. In the embodiment, the first light source 110a and the second light source 110b are spaced apart within the reflective member 120. The reflective member 120 is also provided with a protrusion portion 121 extending toward the gap between the first light source 110a and the second light source 110b. The reflective member 120 is a reflective cup, and the front end of the outlet of the reflective cup is a light emitting channel, and the light emitted from the light source 110 provided in the reflective member 120 can be emitted from the light emitting channel to the light outlet 1201, and then from the light outlet 1201 to the human skin.

In the embodiment, the protrusion portion 121 is a component protruding from the reflective side of the reflective member 120 toward the light outlet 1201. For example, the reflective member 120 includes two opposite reflective surfaces, one reflective surface is provided towards the first light source 110a and another reflective surface is provided towards the second light source 110b. The protrusion portion 121 can protrude toward the light outlet 1201 from any position on either of the two spaced reflective surfaces.

Since related technology places multiple lamps in a single reflective cup, causing light interference between multiple lamps and mutual blocking of reflected light, which leads to weakened light energy output from the lamps, this embodiment of the present application provides a protrusion portion 121 extending into the gap between the first light source 110a and second light source 110b within the reflective member 120, the light generated by the first light source 110a and the second light source 110b within the reflective member 120 will not block each other and the light generated by the first light source 110a and the second light source 110b will not interfere with each other, so that the light energy generated by the first light source 110a and the second light source 110b will not diminish, so that the light energy of the light source assembly 400 can be further improved, this achieves the effect of further enhancing the light output energy of the light source assembly 400, improving beauty treatment effects, and meeting user requirements.

In one embodiment of the present application, as shown in FIGS. 7 and 8, the reflective member 120 includes a reflective side 1202 facing the first light source 110a and second light source 110b, and a heat dissipation side 1203 away from the first light source 110a and second light source 110b. The protrusion portion 121 includes a first reflective surface 1211 and a second reflective surface 1212 disposed on the reflective side 1202, the first reflective surface 1211 and second reflective surface 1212 are arranged opposite to each other. The first reflective surface 1211 and second reflective surface 1212 correspond to the first light source 110a and second light source 110b respectively, and neither reflective surface corresponds to the same light source. The first reflective surface 1211 and second reflective surface 1212 are disposed on opposite sides of the protrusion portion 121. The first reflective surface 1211 faces the first light source 110a, and the second reflective surface 1212 faces the second light source 110b. This embodiment, by setting the first reflective surface 1211 and second reflective surface 1212 on the protrusion portion 121, can increase the reflection area for the first light source 110a and second light source 110b on the reflective member 120. Compared to related technology where multiple light sources are disposed in a single reflective cup, this design of the present application can increase the reflection area for each light source 110, thereby enhancing the light output energy of each light source 110 and achieving increased light output energy.

As shown in FIGS. 7 and 8, the reflective member 121 also includes a third reflective surface 122 and/or a fourth reflective surface 123 disposed on the reflective side 1202, the third reflective surface 122 and fourth reflective surface 123 are spaced apart on the reflective member 120. The third reflective surface 122 corresponds to the first light source 110a, and the fourth reflective surface 123 corresponds to the second light source. One end of the first reflective surface 1211 connects with the third reflective surface 122 and corresponds to the first light source 110a. One end of the second reflective surface 1212 connects with the fourth reflective surface 123 and corresponds to the second light source 110b. Another end of the first reflective surface 1211 away from the third reflective surface 122 connects with another end of the second reflective surface 1212 away from the fourth reflective surface 123. Another end of the first reflective surface 1211 and another end of the second reflective surface 1212 extend toward the gap between the first light source 110a and second light source 110b.

In some embodiments, as shown in FIGS. 7 and 8, the reflective member 120 is provided with a first reflective cavity 124 and/or a second reflective cavity 125. The first reflective surface 1211 connects with the third reflective surface 122 to form the first reflective cavity 124. The second reflective surface 1212 connects with the fourth reflective surface 123 to form the second reflective cavity 125. The first light source 110a is disposed within the first reflective cavity 124, and the second light source 110b is disposed within the second reflective cavity 125. The present application forms multiple reflective cavities by connecting the reflective surface of the protrusion portion 121 with the reflective surface on the reflective member, so that the protrusion portion 121 can prevent the light emitted by the first light source 110a located in the first reflective cavity 124 from being emitted to the second light source 110b located in the second reflective cavity 125, and also prevent light reflected from the first reflective surface 1211 and the third reflective surface 123 from reflecting into the second light source 110b. In the embodiment, the first reflective cavity 124 semi-surrounds the first light-emitting member 110a, and the second reflective cavity 125 semi-surrounds the second light source 110b. The reflective member 120 can include multiple reflective cavities, all of which are semi-surrounding structures, and these multiple reflective cavities can be obtained through connection or integral molding. In the embodiment, using two light sources as an example, the structure consists of two semi-surrounding reflective cavities, forming a W-shaped reflective member. When there are three or more reflective cavities, the structure of the reflective member can be seen as a combination structure connected by multiple semi-surrounding or U-shaped structures, with no specific limitations here.

In an optional embodiment, as shown in FIGS. 7 to 9, the reflective member 120 has a first end 1204 and a second end 1205 corresponding to the first end 1204, the light outlet 1201 is formed between the first end 1204 and second end 1205.

In an optional embodiment, as shown in FIGS. 7 and 8, the reflective member 120 forms a heat dissipation area 1206 on the heat dissipation side 1203, the heat dissipation area 1206 extends from the first end 1204 or the second end 1205 to the protrusion portion 121. In the embodiment, the heat dissipation area 1206 refers to the heat dissipation surface area. Due to the W-shaped design of the reflective member 120, the heat dissipation area 1206 on the heat dissipation side 1203 also follows this W-shape, thereby increasing the heat dissipation surface area and improving the heat dissipation effect of the reflective member 120.

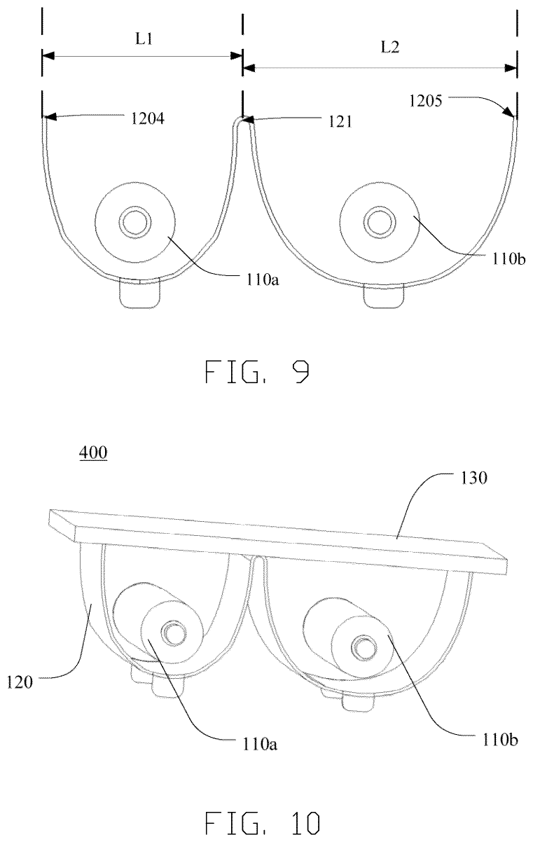

Furthermore, as shown in FIG. 9, the distance from the protrusion portion 121 to the first end 1204 is L1, and the distance from the protrusion portion 121 to the second end 1205 is L2. The ratio of L1 to L2 ranges from 0.2-0.9. In one embodiment, the ratio is 0.2. In another embodiment, the ratio is 0.9; optionally, the ratio is 0.75, in the embodiment, this ratio range is designed to create different light emission areas for the first light source 110a and the second light source 110b. When users select only one light source to work through mode selection, this distance ratio range allows either member to meet different light energy output requirements independently. When multiple light sources 110 work simultaneously, this ratio range enables the combined light energy from multiple members to meet preset requirements for skin treatments like hair removal or whitening, thereby enhancing the output energy and beauty treatment effects of the light source assembly's 400. In addition, the present application sets the ratio range of L1 to L2, when the difference between L1 and L2 is larger, for example, the ratio range is 0.2, the light output area corresponding to the first light source 110a is much smaller than the light output area corresponding to the second light source 110b, the light outlet area of the first light source 110a decreases, and the light emitted by the first light source 110a will be more concentrated, thereby improving the concentration and increasing the energy of the light output; when the difference between L1 and L2 is smaller, for example, the ratio range is 0.9, the light outlet area corresponding to the first light source 110a is not much different from the area light outlet corresponding to the second light source 110b, the light emitted from the first light source 110a and the light emitted from the second light source 110b are more uniform on the corresponding light outlet, and the uniformity of light emitted from the light source assembly is enhanced.

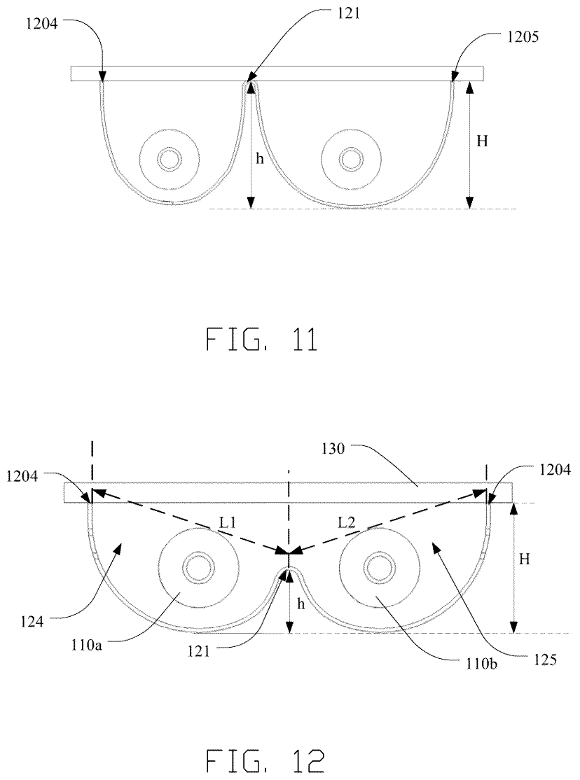

In an optional embodiment, as shown in FIGS. 10 and 11, the light source assembly 400 also includes a light filter 130 disposed on the light outlet 1201, and the light filter 130 is connected to the first end 1204 and the second end 1205. The light filter 130 is a light filter, in some embodiments, the light filter may be obtained through optical coating on an optical substrate using processes such as evaporation deposition, ion beam sputtering (IBS), plasma sputtering, atomic layer deposition (ALD), etc. For example, the light filter may be a single interval band coating, or the light filter may be a dual interval band coating or a zoned coating on the same optical substrate, such as a zoned filter light with zones A and B. In the embodiment, existing technology filter plates can be used. Different skin problems can be treated with different wavelength bands by switching different filter plates, with no specific limitations here.

In the embodiment, the height of the protrusion portion 121 may range from 1 mm-10 mm. In some embodiments, the height may be 1 mm, in another embodiment, the height may be 5 mm, and in another embodiment, the height may be 10 mm.

In the embodiment, the protrusion portion 121 is disposed between the first end 1204 and the second end 1205, the height h of the protrusion portion 121 equals the height H of the second end 1205 extending toward the light outlet 1201. In the embodiment, the height of the first end 1204 extending toward the light outlet 1201 equals the height of the second end 1205 extending toward the light outlet 1201, so either the height of the first end 1204 or the second end 1205 can be used as reference, with no specific limitations here. As shown in FIGS. 10 and 11, when the height h of the protrusion portion 121 equals the height H of the second end 1205 extending toward the light outlet 1201, the protrusion portion 121 abuts against the light filter 130.

When the protrusion portion 121 is abutted against the light filter 130, the protrusion portion 121 may divide the light outlet 1201 into a first light outlet and a second light outlet, the first light outlet corresponding to the first light source 110a and the second light outlet corresponding to the second light source 110b. As shown in FIG. 9, the distance L1 is the length of the first light outlet and the distance L2 is the length of the second light outlet, at this point, the ratio of L1 to L2 can be replaced by the ratio of the length of the first light outlet to the length of the second light outlet.

In the embodiment, the height h of the protrusion portion 121 is not higher than the height H of the second end 1205 extending towards the light outlet 1201, so that the reflective effect of multiple light sources 110 disposed within the reflective member 120 and the light mixing effect between multiple light sources 110 can be adjusted. When there is a need to enhance the reflective effect of the reflective member 120, the height h of the protrusion portion 121 may be set to coincide with the height H of the second end 1205 extending toward the light outlet 1201, so that the reflective surface on the protrusion portion 121 combines with the reflective surface on the reflective member 120, so that the light generated by the light source 110 does not irradiate the position of the other light sources, and thus enhancing the reflective effect of the reflective member 120, thereby enhancing the light output energy of the single light source 110. When there is a need to enhance the light mixing effect of the reflective member 120, the height h of the protrusion portion 121 may be set to be lower than the height H of the second end 1205 extending toward the light outlet 1201, so that the light generated by the first light source 110a is partially irradiated to the position of the second light source 110b, so that the light generated by the second light source 110b is mixed with the light generated by the first light source 110a, thereby enhance the effect of mixing the light output, and thereby enhance the light output energy when multiple light sources 110 work simultaneously.

In some embodiments, the height h of the protrusion portion 121 is lower than the height H of the second end 1205 extending toward the light outlet 1201.

Specifically, as shown in FIG. 12, when the height h of the protrusion portion 121 is less than the height H of the second end 1205 extending toward the light outlet 1201, the protrusion portion 121 does not abut against the light filter 130. The length of the first light outlet corresponding to the first light source 110a is greater than the length from the protrusion portion 121 to the first end 1204 when the protrusion portion 121 is abutted against the light filter 130. In the embodiment, the length of the first light outlet and the second light outlet cannot be divided by the protrusion portion 121, this can be replaced by directly using the ratio of the distance L1 between the protrusion portion 121 and the first end 1204 to the distance L2 between the protrusion portion 121 and the second end 1205.

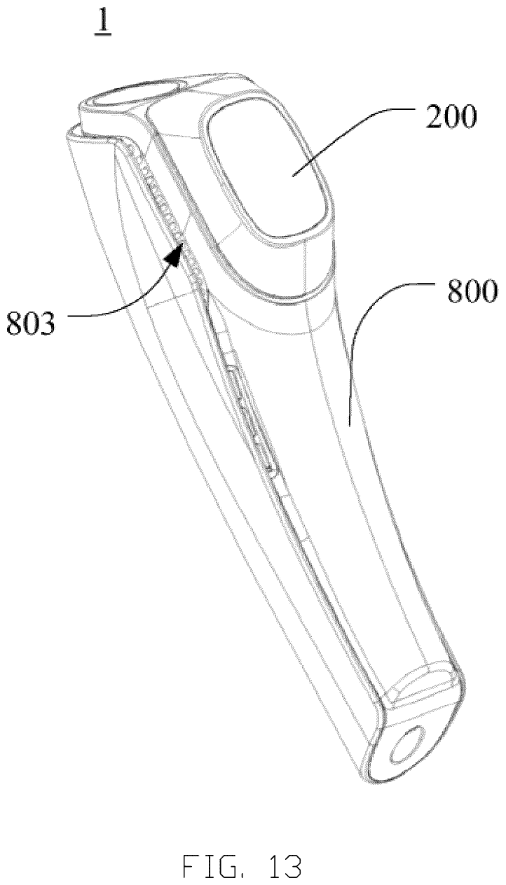

As shown in FIGS. 13 to 15, another embodiment of the present application also provides a skin treatment device 1, the skin treatment device 1 includes a housing 800, a light source assembly 400, and a light transmissive member 200.

The light source assembly 400 is the light source assembly 400 as described above; the light source assembly 400 is disposed within the housing 800. The light transmissive member 200 may be provided opposite to the light source assembly 400, and the light generated by the light source assembly 400 is emitted outwardly through the light transmissive member 200. The light transmissive member 200 may be provided within the housing 800, or part of the light transmissive member 200 is provided exposed to the surface of the housing 800, or the light transmissive member 200 is provided on the surface of the housing 800, the light transmissive member 200 may be disposed within or on the housing 800 according to the actual situation, or partially inside the housing 800, with no specific limitations here.

As shown in FIGS. 13 to 16, the skin treatment device 1 further includes an installation bracket 500 arranged inside the housing 800, the installation bracket 500 has an air duct 506 inside, and the light source assembly 400 is located inside the air duct 506. The housing 800 is provided with a ventilation opening 803, the ventilation opening 803 is communicated with the air duct 506. The present application sets up an installation bracket 500 and places the light source assembly 400 inside the air duct 506, which may dissipate heat from the light source assembly 400 by connecting the ventilation opening 506 with the air duct 506. In the embodiment, the installation bracket 500 may be a shell bracket, which can be composed of an upper shell bracket and a lower shell bracket, and the installation bracket 500 is formed by the connection between the upper shell bracket and the lower shell bracket.

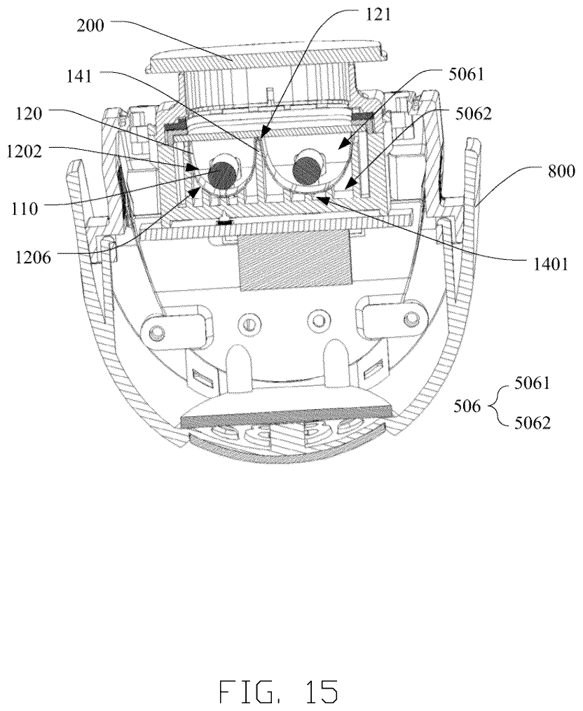

In some embodiments, as shown in FIG. 15, the reflective member 120 includes a reflective side 1202 facing the light source 110 and a heat dissipation side 1203 away from the light source 110. The reflective member 120 divides the air duct 506 into a first air duct 5061 and a second air duct 5062. The first air duct 5061 corresponds to the multiple light sources 110 and the first air duct 5061 is located between the reflective side 1202 and the inner wall of the installation bracket 500. The second air duct 5062 is located between the heat dissipation side 1203 and the inner wall of the installation bracket 500. In the embodiment, the inner wall of the installation bracket 500 includes a bottom inner wall, a top inner wall opposite to the bottom inner wall and left and right inner walls arranged between the bottom and top inner walls. The first air duct 5061 is located between the reflective side 1202 and the top inner wall of the installation bracket 500, and the second air duct 5062 is located between the heat dissipation side 1203 and the bottom inner wall of the installation bracket 500. In other words, the reflective member 120 may divide the air duct 506 into an upper air duct and a lower air duct, the first air duct 5061 is the upper air duct and the second air duct 5062 is the lower air duct.

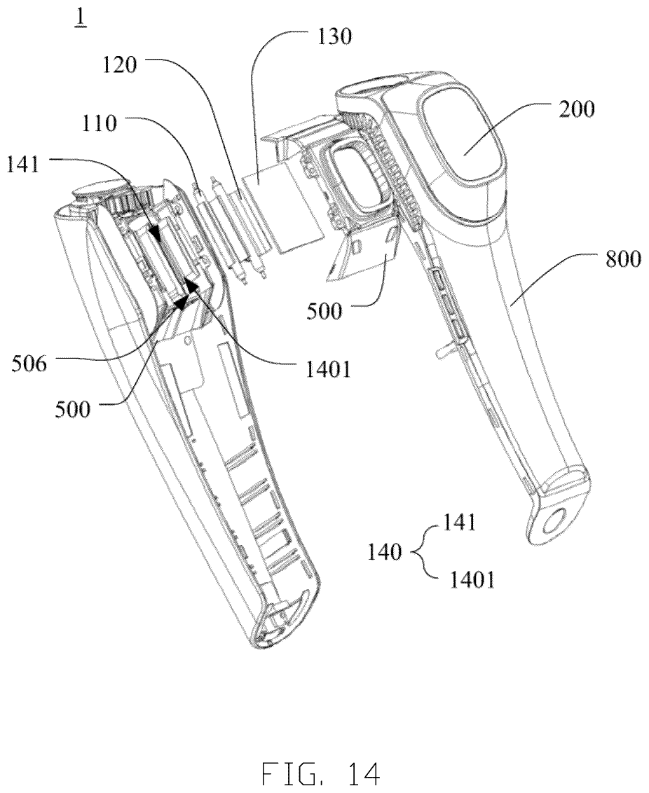

In some embodiments, as shown in FIGS. 14 and 15, the skin treatment device 1 further includes a heat dissipation portion 140 located inside the second air duct 5062, and the heat dissipation portion 140 is used to dissipate the heat of the heat dissipation side 1203.

Specifically, the heat dissipation portion 140 forms multiple heat dissipation gaps 1401 in the second air duct 5062, the multiple heat dissipation gaps 1401 are used to dissipate the heat from the heat dissipation side 1203.

Furthermore, the heat dissipation side 1203 is formed with a heat dissipation area 1206. The heat dissipation portion 140 includes a support member 141, the support member 141 is located between the heat dissipation side 1203 and the bottom inner wall of the installation bracket 500. The support members 141 are multiple, and any two adjacent support members 141 and the heat dissipation area 1206 can jointly form the heat dissipation gap 1401. When the heat generated by the light source 110 within the reflective member 120 reaches the heat dissipation side 1203 of the reflective member 120, the heat generated by the light source may be dissipated by the heat dissipation gap 1401, thereby achieving the purpose of dissipating heat to the reflective member 120.

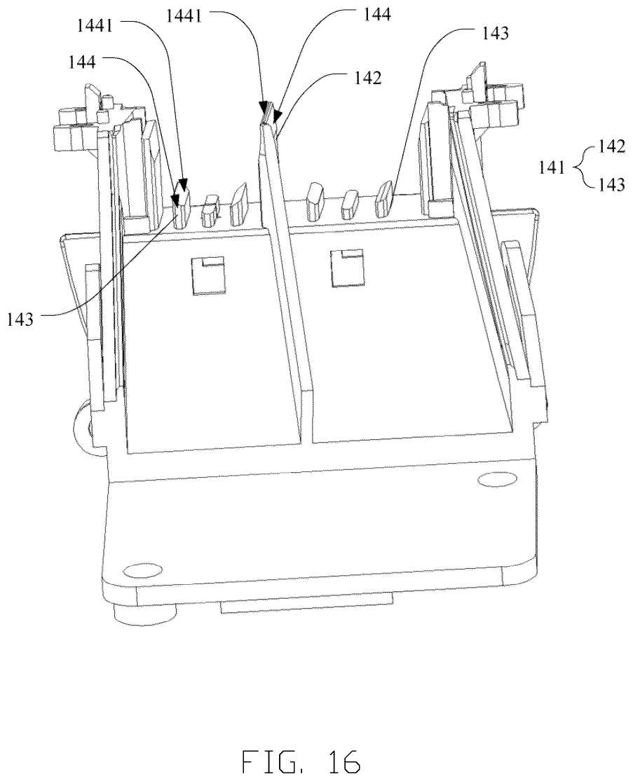

As shown in FIG. 16, the heat dissipation portion 140 also includes a support member 141, the support member 141 includes a first support member 142 and multiple second support members 143, multiple second support members 143 are distributed on both sides of the first support member 142, and the first support member 142 is connected to the protrusion portion 121. The height of the second support member 143 is less than the height of the first support member 142. The second support member 143 is connected to other areas on the reflective member 120 except for the protrusion portion 121.

Furthermore, as shown in FIGS. 15 and 16, the first support member 142 and the second support member 143 are adapted to the reflective member 120. Both the first support member 142 and the second support member 143 include an end portion 144 proximate the reflective member 120, the end portion 144 is formed with a curved surface 1441, the curved surface 1441 is adapted to the reflective member 120. As can be seen from the above first embodiment, the reflective member 120 in this embodiment is a reflective member 120 of a W-shaped structure, and the heat dissipation side 1203 on the reflective member 120 is provided in a curved or semi-circular or semi-elliptical shape. For the first support member 142 and the second support member 143 to be adapted to the reflective member 120, the end surfaces on the end portions of the first support member 142 and the second support member 143 proximate to the end portion of the reflective member 120 must be in contact with the heat dissipation side 1203 on the reflective member 120. When the heat dissipation side 1203 on the reflective member 120 is provided in a curved or semi-circular or semi-elliptical shape, the end surface formed on the end portion is a curved surface 1441. As can be seen from FIG. 16, the second support members 143 on the left of the first support member 142 are spaced apart, the curved surface of each second support member 143 is different because the contact position between the second support member 143 and the reflective member 120 is different. When the structure of the reflective member 120 is arranged in a parabolic shape, the arrangement of the curved surface 1441 in the second support member 143 is an arc-shaped arrangement that first descends and then rises. The curved shape of the curved surface 1441 in the present application is changed according to the change of the specific shape structure of the reflective member 120. By such a setting in the present application, the support member 141 and the reflective member 120 are made to achieve a perfectly adapted connection, so as to realize the effect of fixation and heat dissipation and improve the assembly efficiency and heat dissipation efficiency of the skin treatment device 1.
Clause 1. a light source assembly applied to a skin treatment device, comprising:
multiple light sources; and
a reflective member having a light outlet, the light outlet is configured for emitting light from the multiple light sources;
wherein any two adjacent light sources of the multiple light sources are spaced apart within the reflective member; and wherein the reflective member is further provided with a protrusion portion extending toward a gap between the any two adjacent light sources.
Clause 2. the light source assembly according to clause 1, wherein the reflective member comprises a reflective side facing the multiple light sources and a heat dissipation side away from the multiple light sources, the protrusion portion comprises a first reflective surface and a second reflective surface disposed on the reflective side, and the first reflective surface is disposed opposite to the second reflective surface.
Clause 3. the light source assembly according to clause 2, wherein the light source comprises a first light source and a second light source adjacent to and spaced apart from the first light source; the reflective member further comprises a third reflective surface and/or a fourth reflective surface arranged on the reflective side, the third reflective surface is spaced apart from the fourth reflective surface on the reflective member, the third reflective surface corresponds to the first light source, and the fourth reflective surface corresponds to the second light source; one end of the first reflective surface is connected to the third reflective surface and corresponds to the first light source; one end of the second reflective surface is connected to the fourth reflective surface and corresponds to the second light source.
Clause 4. the light source assembly according to clause 3, wherein the reflective member is provided with a first reflective cavity and/or a second reflective cavity, and the first reflective surface is connected to the third reflective surface to form the first reflective cavity; the second reflective surface is connected to the fourth reflective surface to form the second reflective cavity; the first reflective cavity is provided with the first light source; the second reflective cavity is provided with the second light source.
Clause 5. the light source assembly according to any one of clauses 1 to 4, wherein the reflective member comprises a first end and a second end corresponding to the first end, and a light outlet is formed between the first end and the second end.
Clause 6. the light source assembly according to clause 5, wherein the protrusion portion is disposed between the first end and the second end, and a height of the protrusion portion does not exceed a height of the first end and/or the second end extending towards the light outlet.
Clause 7. the light source assembly according to clause 5 or clause 6, wherein the light source assembly further comprises a light filter, the light filter is disposed on the light outlet, and the light filter is connected to the first end and the second end.
Clause 8. the light source assembly according to clause 7, wherein when the height of the protrusion is lower than the height of the first end and/or the second end extending towards the light outlet, the protrusion does not abut against the light filter; or when the height of the protrusion portion is equal to the height of the first end and/or the second end extending towards the light outlet, the protrusion portion abuts against the light filter.
Clause 9. the light source assembly according to any one of clauses 1 to 8, wherein the height of the protrusion portion is 1 mm-10 mm.
Clause 10. the light source assembly according to any one of clauses 5 to 9, wherein the reflective member includes a heat dissipation area on the heat dissipation side, and the heat dissipation area extends from the first end or the second end to the protrusion portion.
Clause 11. the light source assembly according to any one of clauses 6 to 10, wherein a ratio range of a first distance between the protrusion portion and the first end to a second distance between the protrusion portion and the second end is 0.2-0.9.
Clause 12. a skin treatment device comprising:
a housing;
the light source assembly as claimed in any one of clauses 1-11, wherein the light source assembly is disposed inside the housing; and
a light transmissive member is corresponded to the light source assembly, and light from the light source assembly is emitted outward through the light transmissive member.
Clause 13. the skin treatment device according to clause 12, wherein the skin treatment device further comprises an installation bracket disposed inside the housing, the installation bracket has an air duct, and the light source assembly is located inside the air duct; the housing is provided with a ventilation opening, and the ventilation opening is communicated with the air duct.
Clause 14. the skin treatment device according to clause 13, wherein the reflective member comprises a reflective side facing the multiple light sources and a heat dissipation side away from the multiple light sources; the reflective member divides the air duct into a first air duct and a second air duct; the first air duct corresponds to the multiple light sources and the first air duct is located between the reflective side and an inner wall of the installation bracket, the second air duct is located between the heat dissipation side and the inner wall of the installation bracket.
Clause 15. the skin treatment device according to clause 14, wherein the skin treatment device further comprises a heat dissipation portion, the heat dissipation portion is located inside the second air duct, and the heat dissipation portion is configured to dissipate heat on the heat dissipation side.
Clause 16. the skin treatment device according to clause 15, wherein the heat dissipation portion forms multiple heat dissipation gaps in the second air duct, the multiple heat dissipation gaps are configured to dissipate heat on the heat dissipation side.
Clause 17. the skin treatment device according to clause 16, wherein the heat dissipation side is formed with a heat dissipation area, and the heat dissipation portion comprises multiple support members located between the heat dissipation side and the inner wall of the installation bracket, and any two adjacent support members form a heat dissipation gap together with the heat dissipation area.
Clause 18. the skin treatment device according to any one of clauses 15 to 17, wherein the heat dissipation portion further comprises a support member, the support member comprises a first support member and multiple second support members, the second support members are distributed on both sides of the first support member, and the first support member is connected to the protrusion portion; a height of the second support member is less than a height of the first support member.
Clause 19. the skin treatment device according to clause 18, wherein the first support member and the second support member are adapted to the reflective member; the first support member and the second support member both include an end portion close to the reflective member, and a curved surface is formed on the end portion, and the curved surface is adapted to the reflective member.

In the present application, unless otherwise specified or limited, terms such as “installation” and “connection” should be interpreted broadly. For example, it can be a fixed connection, a detachable connection, or an integrated connection; it can be a mechanical connection; It can be a direct connection, an indirect connection through an intermediate medium, an internal connection between two components, a surface contact only connection, or a surface contact connection through an intermediate medium. For ordinary technical personnel in this field, the specific meanings of the above terms in the present application can be understood according to the specific situation.

In addition, the terms “first”, “second”, etc. are used only to distinguish descriptions and are not to be construed as referring to a specific structure. The description of the term “some embodiments” means that specific features, structures, materials, or characteristics described in connection with the embodiment or example are included in at least one embodiment or example of the present application. For the purposes of the present application, schematic representations of the above terms need not be directed to the same embodiments or examples. Moreover, the specific features, structures, materials, or characteristics described may be combined in any one or more embodiments or examples in a suitable manner. Furthermore, without contradicting each other, those skilled in the art may combine and combine different embodiments or examples and features of different embodiments or examples described in the present application.

The above embodiments are only used to illustrate the technical solutions of the present application, not to limit them; although the present application has been described in detail with reference to the foregoing embodiments, a person of ordinary skill in the art should understand that it is still possible to make modifications to the technical solutions documented in the foregoing embodiments, or to make equivalent substitutions for some of the technical features therein; and such modifications or substitutions do not detract from the essence of the corresponding technical solutions. The spirit and scope of the technical solutions of the embodiments of the present application shall be included in the scope of protection of the present application.

1. A skin treatment device comprising:
a housing comprising a light emitting portion; and
a light emitting assembly disposed in the housing; wherein a light emitting channel is formed between the light emitting assembly and the light emitting portion, the light emitting channel is provided with a partition member, the partition member divides the light emitting channel into a first sub-light emitting channel and a second sub-light emitting channel, both the first sub-light emitting channel and the second sub-light emitting channel are opposite to the light emitting portion, the light emitting assembly comprises a first light source and a second light source, the first light source is opposite to the first sub-light emitting channel and configured to generate light that emits toward the light emitting portion through the first sub-light emitting channel, the second light source is opposite to the second sub-light emitting channel and configured to generate light that emits toward the light emitting portion through the second sub-light emitting channel; a type of the first light source is different from a type of the second light source,
wherein the first light source is a halogen light source, and the second light source is a pulse light source, and the first light source and the second light source are configured to provide targeted treatment effects for different skin problems.

2. The skin treatment device according to claim 1, wherein a light guiding ring is disposed within the light emitting channel, the partition member is located in an inner ring space of the light guiding ring, the partition member comprises a first surface and a second surface, the first surface and the second surface face away from each other, the first sub-light emitting channel is located between an inner ring surface of the light guiding ring and the first surface, and the second sub-light emitting channel is located between the inner ring surface of the light guiding ring and the second surface, and preferably,
wherein a reflective layer is disposed on the inner ring surface of the light guiding ring, and the reflective layer is located in the first sub-light emitting channel and/or the second sub-light emitting channel.

3. The skin treatment device according to claim 2, wherein the first surface and/or the second surface is provided with a reflective layer.

4. The skin treatment device according to claim 1, wherein the light emitting assembly comprises a light filter, the light filter is disposed in light paths of the first light source and the second light source, the partition member abuts against a surface of the light filter that faces away from the first light source or the second light source.

5. The skin treatment device according to claim 4, wherein the partition member extends from the light filter to the light emitting portion, and/or
wherein the light emitting portion is provided with a cold compress member for cold compressing the skin, the cold compress member is located in the light paths of the first light source and the second light source, one side of the partition member backing away from the light filter is abutted against the cold compress member, or there is a gap between one side of the partition member backing away from the light filter and the cold compress member.

6. The skin treatment device according to claim 4, wherein the light guiding ring is abutted against or has a gap with a surface of the light filter back away from the first light source or the second light source; and/or
the light emitting portion is provided with a cold compress member for cold compressing the skin, the cold compress member is located in the light paths of the first light source and the second light source, one side of the light guiding ring backing away from the light filter is abutted against the cold compress member, or there is a gap between one side of the light guiding ring backing away from the light filter and the cold compress member.

7. The skin treatment device according to claim 4, wherein the skin treatment device further comprises a supplementary light source, and the supplementary light source is disposed within the housing, there is a gap between the light guiding ring and the light filter, and light from the supplementary light source is emitted through the gap to the light emitting channel, and preferably,
wherein the supplementary light source surrounds the light emitting channel, and preferably, wherein the partition member comprises a support portion, the support portion extends into the gap, one end of the support portion abuts against the light filter, another end of the support portion abuts against the light guiding ring, thereby maintaining the gap between the light guiding ring and the light filter.

8. The skin treatment device according to claim 2, wherein the light guiding ring and the partition member are an integrally molded structure.

9. The skin treatment device according to claim 5, wherein the skin treatment device further comprises a refrigerator, a cooling surface of the refrigerator is provided on a surface of the cold compress member toward the light emitting assembly, and the refrigerator is located on an outer periphery of the first sub-light emitting channel or the second sub-light emitting channel.

10. The skin treatment device according to claim 1, wherein the light emitting assembly comprises a reflective member, the reflective member has a light outlet, the light outlet is configured for emitting light from the first light source and the second light source; wherein the first light source and the second light source are spaced apart within the reflective member; and the reflective member is further provided with a protrusion portion extending toward a gap between the first light source and the second light source.

11. The skin treatment device according to claim 10, wherein the reflective member comprises a reflective side facing the first light source and the second light source and a heat dissipation side away from the first light source and the second light source, the protrusion portion comprises a first reflective surface and a second reflective surface disposed on the reflective side, and the first reflective surface is arranged opposite to the second reflective surface.

12. The skin treatment device according to claim 11, wherein the light source comprises a first light source and a second light source adjacent to and spaced apart from the first light source; the reflective member further comprises a third reflective surface and/or a fourth reflective surface arranged on the reflective side, the third reflective surface is spaced apart from the fourth reflective surface on the reflective member, the third reflective surface corresponds to the first light source, and the fourth reflective surface corresponds to the second light source; one end of the first reflective surface is connected to the third reflective surface and corresponds to the first the first light source; one end of the second reflective surface is connected to the fourth reflective surface and corresponds to the second light source.

13. The skin treatment device according to claim 12, wherein the reflective member is provided with a first reflective cavity and/or a second reflective cavity, and the first reflective surface is connected to the third reflective surface to form the first reflective cavity; the second reflective surface is connected to the fourth reflective surface to form the second reflective cavity; the first reflective cavity is provided with the first light source; the second reflective cavity is provided with the second light source.

14. The skin treatment device according to claim 10, wherein the reflective member comprises a first end and a second end corresponding to the first end, and a light outlet is formed between the first end and the second end.

15. The skin treatment device according to claim 14, wherein the protrusion portion is disposed between the first end and the second end, and a height of the protrusion portion does not exceed a height of the first end and/or the second end extending towards the light outlet.

16. The skin treatment device according to claim 14, wherein the light source assembly further comprises a light filter, the light filter is disposed on the light outlet, and the light filter is connected to the first end and the second end.

17. The skin treatment device according to claim 16, wherein when the height of the protrusion is lower than the height of the first end and/or the second end extending towards the light outlet, the protrusion does not abut against the light filter; or when the height of the protrusion portion is equal to the height of the first end and/or the second end extending towards the light outlet, the protrusion portion abuts against the light filter.

18. The skin treatment device according to claim 10, wherein the height of the protrusion portion is 1 mm-10 mm.

19. The skin treatment device according to claim 14, wherein the reflective member includes a heat dissipation area on the heat dissipation side, and the heat dissipation area extends from the first end or the second end to the protrusion portion.

20. The skin treatment device according to claim 15, wherein a ratio range of a first distance between the protrusion portion and the first end to a second distance between the protrusion portion and the second end is 0.2-0.9.
